(19) United States
(12) Patent Application Publication
(10) Pub. No.: US 2026/0269043 A1
(43) Pub. Date: Sep. 10, 2026

(54) INFUSION SYSTEM AND STAGED INFUSION METHOD THEREOF

(71) Applicants: Inventec Appliances (Pudong) Corporation, Shanghai (CN); Inventec Appliances (Shanghai) Co.,Ltd., Shanghai (CN); Inventec Appliances Corp., New Taipei City (TW); Inventec Appliances (Nanchang) Corporation, Jiangxi (CN)

(72) Inventors: Shieng-Ting KAO, New Taipei City (TW); Hao-Ming LI, New Taipei City (TW)

(21) Appl. No.: 18/851,225

(22) PCT Filed: Feb. 4, 2024
(86) PCT No.: PCT/CN2024/075819

§ 371 Date: Sep. 26, 2024

(51) Int. Cl. G16H 20/17; A61M 5/172; G16H 10/60

(52) U.S. Cl. CPC G16H 20/17; A61M 5/172; G16H 10/60; A61M 2205/502

(57) ABSTRACT

An infusion system including a receiving module, a server, a transmission module and an infusion device is provided. The receiving module receives a patient information and a corresponding prescription information from a medical system, wherein the prescription information includes at least one fluid name, at least one fluid dose and a staged infusion parameter, and the patient information includes a patient name and a corresponding position of a hospital bed. The server transmits the prescription information to the infusion device located at the hospital bed according to the patient information. The infusion device displays the prescription information and the patient information and performs a staged infusion according to the staged infusion parameter.

This application is the 35 U.S.C. § 371 national stage of PCT application PCT/CN 2024/075819, filed Feb. 4, 2024, the disclosure of which is hereby incorporated by reference.

### BACKGROUND OF THE INVENTION

### Field of the Invention

The invention relates in general to an infusion management technique, and more particularly to an infusion system and a staged infusion method thereof.

### Description of the Related Art

Generally speaking, medical staff can adjust infusion volume according to the prescription. According to the conventional method of adjusting infusion volume, medical staff needs to manually operate the infusion device. Since the conventional method is susceptible to the risk of human errors, other medical staff need to double check to avoid errors. Furthermore, for some patients, several items of infusion data need to be inputted during the course of treatment. Thus, to be responsible for the patients'health, there exists an urgent need to improve the operation of infusion.

### SUMMARY OF THE INVENTION

The invention is directed to an infusion system and a staged infusion method thereof. The staged infusion parameters recorded in the prescription are directly inputted to the infusion device, so that the risk of having human errors during the manual input of infusion parameters can be reduced.

According to one embodiment of the present invention, an infusion system is provided. The infusion system includes a receiving module, a server, a transmission module and an infusion device. The receiving module receives a patient information and a corresponding prescription information from a medical system, wherein the prescription information includes at least one fluid name, at least one fluid dose and a staged infusion parameter; the patient information includes a patient name and a corresponding position of a hospital bed. The server is connected to the receiving module to receive the prescription information and the patient information from the receiving module. The transmission module serially connects the server and the infusion device and communicates with the server and the infusion device, so that the server transmits the prescription information to the infusion device located at the hospital bed according to the patient information. The infusion device displays the prescription information and the patient information and performs a staged infusion according to the staged infusion parameter.

According to another embodiment of the present invention, a staged infusion method configured in an infusion system is provided. The staged infusion method includes the following steps. A patient information and a corresponding prescription information are received from a medical system by the infusion system, wherein the prescription information includes at least one fluid name, at least one fluid dose and a staged infusion parameter; the patient information includes a patient name and a corresponding position of a hospital bed. The prescription information is transmitted to an infusion device located at the hospital bed by the infusion system according to the patient information, wherein the infusion device displays the prescription information and the patient information. After the prescription information and the patient information are verified by a medical staff, the infusion device is informed to perform a staged infusion according to the staged infusion parameter.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 is a schematic diagram of an infusion system according to an embodiment of the present invention.

FIG. 2 is a tree diagram of a patient information and a prescription information.

FIG. 3 is a flowchart of a staged infusion method according to an embodiment of the present invention.

FIG. 4 is a detailed flowchart of each step of staged infusion.

FIG. 5 is an embodiment of staged infusion.

FIG. 6 is another embodiment of staged infusion.

FIG. 7 is another embodiment of staged infusion.

### DETAILED DESCRIPTION OF THE INVENTION

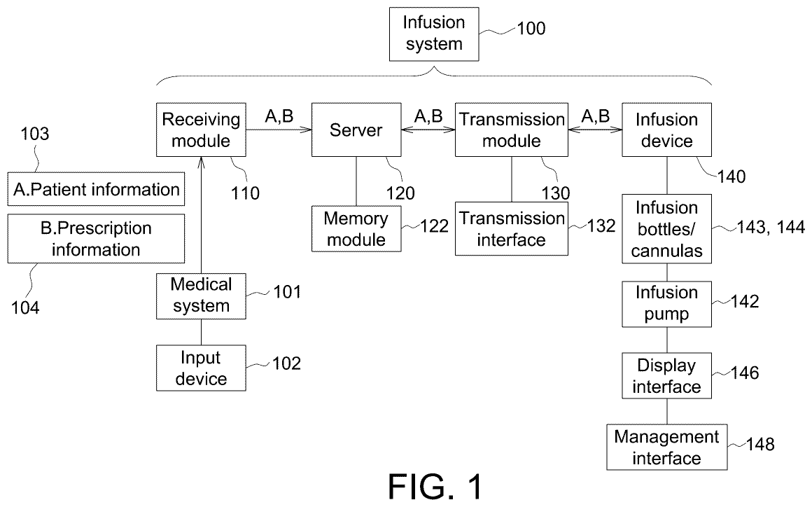

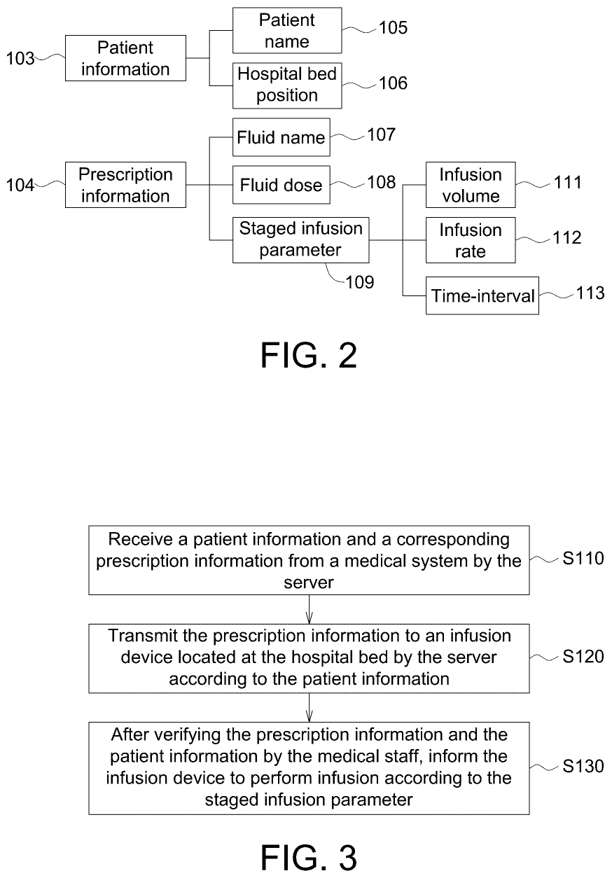

Refer to FIGS. 1 to 3. FIG. 1 a schematic diagram of an infusion system 100 according to an embodiment of the present invention. FIG. 2 is a tree diagram of a patient information 103 and a prescription information 104. FIG. 3 is a flowchart of a staged infusion method according to an embodiment of the present invention. The staged infusion method of FIG. 3 can be implemented by the infusion system 100 of FIG. 1.

Refer to FIG. 1 and FIG. 2. The infusion system 100 includes a receiving module 110, a server 120, a transmission module 130 and an infusion device 140. The receiving module 110, which can be a Bluetooth receiver, a Wi-Fi receiver, or other types of wireless receiver, receives a patient information 103 and a corresponding prescription information 104 from a medical system 101. The server 120, which can be realized by a web host or a database server, provides file management, data storage, and editing functions. The server 120 may include elements such as central processing unit, hard disc, and memory. The server 120 connects the receiving module 110 to receive the prescription information 104 and the patient information 103 from the medical system 101 then store the data to the memory module 122 (such as hard disc or memory).

The transmission module 130, which can be a wireless transceiver and a wireless communication module provided with a transmission interface 132 capable of supporting Bluetooth, Wi-Fi or other communication protocols, serially connects the server 120 and the infusion device 140 and communicates with the server 120 and the infusion device 140 to increase data transfer rate. Besides, the receiving module 110 and the transmission module 130 can transmit data using a transmission line (such as fiber optic or cable) and an Ethernet network, and do not have to adopt a wireless network or a wireless transceiver.

The infusion device 140, such as a relevant device of intravenous injection, may include an infusion pump 142, a plurality of infusion bottles 143, a plurality of infusion cannulas 144, a management interface 148 configured to control the infusion rate and infusion flow, and a display interface 146 configured to display the prescription information 104 and the patient information 103. The management interface 148 can use a computer device to stabilize fluid dispensation, record, track and manage the dose, and emit a warning sound to inform the medical staff in a state of emergency.

According to the conventional method, during the process of intravenous infusion, the medical staff needs to manually set infusion parameters according to the prescription; each hour, the medical staff further need to monitor whether the function of intravenous infusion is normal and whether the infusion rate is correct. The above tasks consume a large amount of labor and cost. In the present embodiment, the server 120 performs smart control on the entire infusion system 100, so that manual operation of the infusion device 140 by the medical staff can be avoided and labor cost can be reduced. The server 120 directly inputs the staged infusion parameter 109 of the prescription information 104 to the infusion device 140, hence avoiding the risk of having human error during the manual input of infusion parameters.

Refer to FIG. 2. The patient information 103 includes a patient name 105 and a corresponding position of a hospital bed 106. The prescription information 104 includes at least one fluid name 107, at least one fluid dose 108 and a staged infusion parameter 109. The staged infusion parameter 109 may include at least one infusion volume 111, at least one infusion rate 112 and a time-interval 113 between any two staged infusions. The infusion volume 111 is equivalent to the product of the infusion rate 112 multiplied by the infusion time.

According to the patient's symptoms, the doctor can prescribe a prescription, and further list the fluid name 107, the fluid dose 108 and the staged infusion parameter 109 in the prescription information 104. For instance, in FIG. 1, the doctor can login to the medical system 101 through the input device 102 to record the patient information 103 and the prescription information 104 in the medical system 101. Besides, the medical system 101 can be transmitted to the receiving module 110 through a wireless module to upload the patient information 103 and the prescription information 104 to the server 120. The input device 102 can be a personalized handheld electronic device (such as a mobile phone, a laptop computer or a tablet PC) or a terminal device (such as a desktop computer).

As indicated in FIG. 3, a patient information 103 and a corresponding prescription information 104 are received from the medical system 101 by the server 120 (step S110), and the prescription information 104 is transmitted to the infusion device 140 located at a hospital bed 106 by the server 120 according to the patient information 103 (step S120). After the prescription information 104 and the patient information 103 are verified, the infusion device 140 is notified by the medical staff t to perform a staged infusion according to the staged infusion parameter 109 (step S130). Thus, the medical staff cannot need to manually set the infusion parameters according to prescription. Furthermore, the infusion system 100 uses the server 120 to automatically monitor whether the function of intravenous infusion is normal or the infusion volume 111 and the infusion rate 112 are correct, so that labor cost can be saved and the workload can be reduced.

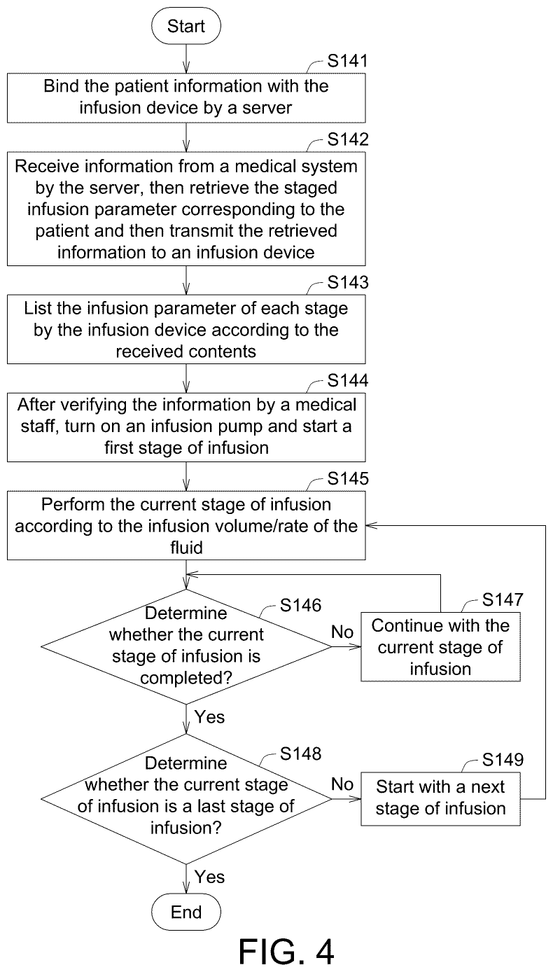

In an embodiment, the staged infusion parameter 109 may include the infusion parameter of multiple stages. Here below, each step of staged infusion is exemplified in an embodiment. Referring to FIG. 4, explanations of the steps S141 to S149 of staged infusion are provided. First, the patient information 103 is bound with the infusion device 140 by a server 120 (step S141). Next, information (such as the patient information 103 and the prescription information 104) are received from a medical system 101 by the server 120, and the staged infusion parameter 109 corresponding to the patient is retrieved and then transmitted to an infusion device 140 (step S142). Then, the infusion parameter of each stage is listed by the infusion device 140 according to the received contents (i.e., the prescription information 104) (step S143). After the information is verified by a medical staff, an infusion pump 142 is turned on, and a first stage of infusion is started (step S144). Then, the current stage of infusion is performed according to the infusion parameter of the current stage (i.e., fluid and infusion volume/rate) (step S145). Then, whether the current stage of infusion is completed is determined (step S146). If the current stage of infusion is not completed, the current stage of infusion continues (step S147); if the current stage of infusion is already completed, whether the current stage of infusion is a last stage of infusion is determined (step S148). If the current stage of infusion is not the last stage of infusion, a next stage of infusion starts (step S149) and the process returns to step S145; if the current stage is the last stage of infusion, then the infusion process terminates.

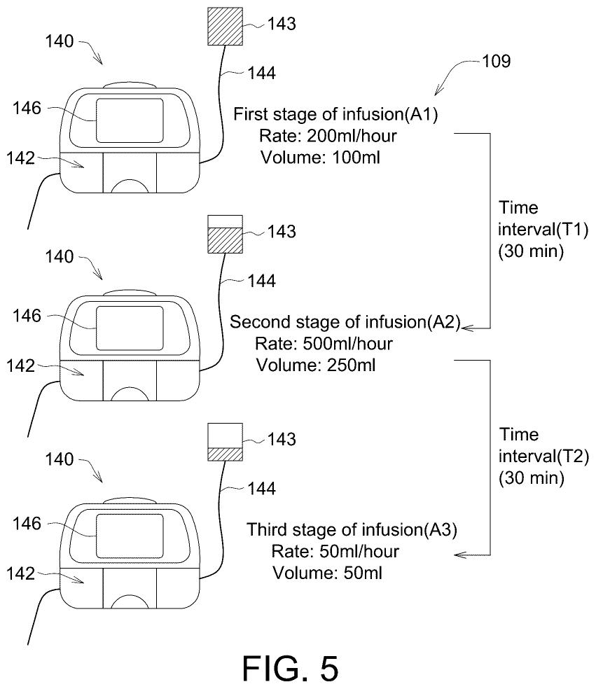

Referring to FIG. 5, an embodiment of staged infusion is provided. In FIG. 5, the staged infusion parameter 109 includes, for instance, a first staged infusion parameter A1, a second staged infusion parameter A2 and a third staged infusion parameter A3. Besides, the staged infusion parameter 109 may further include time-intervals T1 and T2 existing between any two stages of infusion. In an embodiment, the first staged infusion parameter A1 includes a first infusion volume and a first infusion rate; the second staged infusion parameter A2 includes a second infusion volume and a second infusion rate; and the third staged infusion parameter A3 includes a third infusion volume and a third infusion rate. The first time-interval T1 exists between the first stage and the second staged infusion, and the second time-interval T2 exists between the second stage and the third staged infusion.

Each of the first to the third infusion volume is within a range of 50 to 250 milliliters. Each of the first to the third infusion rate is within a range of 50 to 500 milliliters per hour (ml/hour). Each of the first and the second time-intervals T1 and T2 is within a range of 10 to 30 minutes. Of each staged infusion parameter, the infusion volume 111, the infusion rate 112 and the time-interval 113 can be suitably adjusted according to the prescription information 104 and curative effect. If the doctor determines that there is no need to have a time-interval between two stages, the time-interval 113 can be set to 0 in the prescription information 104, so that when one stage of infusion is completed, the process immediately proceeds to the next stage of infusion, and the present invention does not have specific restrictions in this regard.

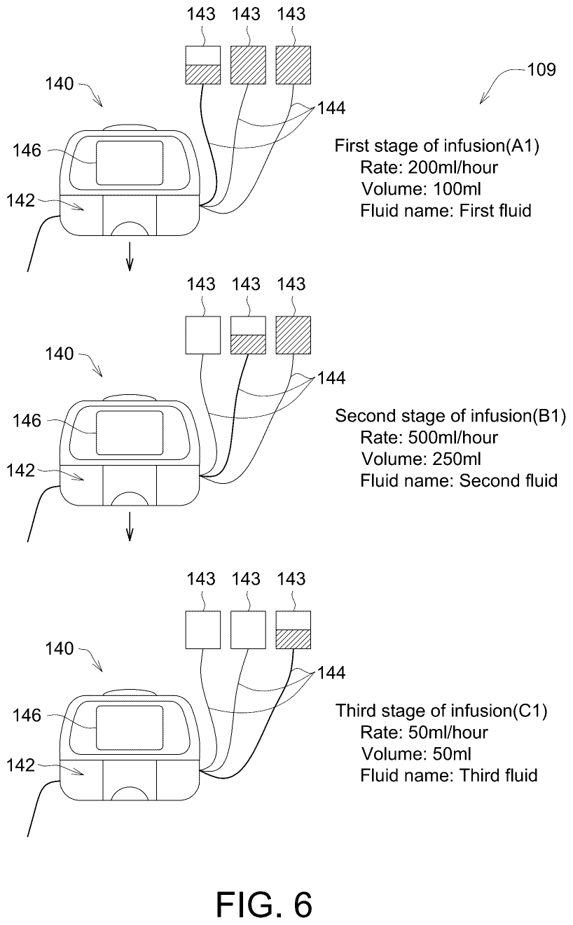

Referring to FIG. 6, another embodiment of staged infusion is provided. In FIG. 6, the staged infusion parameter 109 includes, for instance, a first staged infusion parameter A1, a second staged infusion parameter B1 and a third staged infusion parameter C1. The first staged infusion parameter A1 corresponds to the first fluid; the second staged infusion parameter B1 corresponds to the second fluid; and the third staged infusion parameter C1 corresponds to the third fluid. The first fluid, the second fluid and the third fluid can have different compositions. In an embodiment, the first staged infusion parameter A1 includes the first infusion volume and the first infusion rate corresponding to the first fluid; the second staged infusion parameter B1 includes the second infusion volume and the second infusion rate corresponding to the second fluid; and the third staged infusion parameter C1 includes the third infusion volume and the third infusion rate corresponding to the third fluid.

As indicated in FIG. 6, the infusion device 140 may include an infusion pump 142, a plurality of infusion bottles 143 and a plurality of infusion cannulas 144. Take three infusion bottles 143 and three infusion cannulas 144 for instance. Each infusion bottle 143 contains a fluid, and each infusion cannula 144 correspondingly connects an infusion bottle 143. The infusion pump 142 selectively connects one of the infusion cannulas 144 to perform staged infusion with one of the fluids. For instance, the infusion device 140 firstly performs a first stage of infusion with the first fluid; after a period of time, the infusion device 140 then performs a second stage of infusion with the second fluid; and then after a period of time, the infusion device 140 performs a third stage of infusion with the third fluid.

Each of the first to the third infusion volumes is within a range of 50 to 250 milliliters. Each of the first to the third infusion rates is within a range of 50 to 500 milliliter/hour (ml/hour). Of each staged infusion parameter, the infusion volume 111, the infusion rate 112, the fluid sequence and the time-interval 113 can be suitably adjusted according to the prescription information 104 and curative effect, and the present invention does not have specific restrictions in this regard.

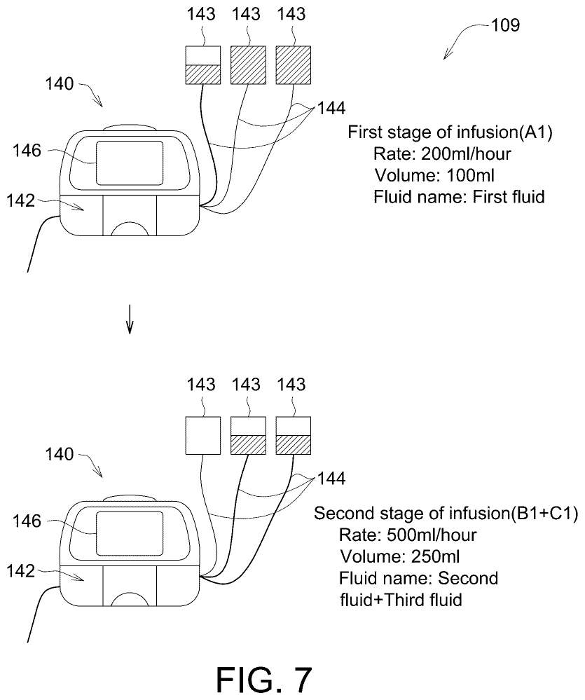

Referring to FIG. 7, another embodiment of staged infusion is provided. In FIG. 7, the staged infusion parameter 109 includes, for instance, a first staged infusion parameter A1 and a second staged infusion parameter B1+C1. The first staged infusion parameter A1 corresponds to the first fluid; the second staged infusion parameter B1+C1 corresponds to the second fluid and the third fluid. The first fluid, the second fluid and the third fluid can have different compositions. The first staged infusion parameter A1 includes a first infusion volume and a first infusion rate corresponding to the first fluid, and the second staged infusion parameter B1+C1 includes a second infusion volume and a second infusion rate corresponding to the second fluid and the third fluid.

In another embodiment not illustrated in the diagram, the first staged infusion parameter corresponds to the first fluid and the second fluid, and the second staged infusion parameter corresponds to the third fluid, and the rest can be obtained by the same analogy.

As indicated in FIG. 7, the infusion device 140 may include an infusion pump 142, a plurality of infusion bottles 143 and a plurality of infusion cannulas 144. Take three infusion bottles 143 and three infusion cannulas 144 for instance. Each infusion bottle 143 contains a fluid, and each infusion cannula 144 correspondingly connects an infusion bottle 143. The infusion pump 142 selectively connects one or more than one of the infusion cannulas 144 to perform staged infusion with one or more than one of the fluids. For instance, the infusion device 140 firstly performs a first stage of infusion with the first fluid. After a period of time, the infusion device 140 then performs a second stage of infusion with the second fluid and the third fluid.

Each of the first to the second infusion volumes is within a range of 100 to 250 milliliters (ml). Each of the first to the third infusion rates is within a range of 200 to 500 milliliters per hour (ml/hour). Of each staged infusion parameter, the infusion volume 111, the infusion rate 112, the fluid sequence and the time-interval 113 can be suitably adjusted according to the prescription information 104 and curative effect, and the present invention does not have specific restrictions in this regard.

In another embodiment not illustrated in the diagram, the infusion device 140 may include a plurality of infusion pumps 142, a plurality of infusion bottles 143 and a plurality of infusion cannulas 144. Each infusion pump 142 correspondingly connects an infusion cannula 144 and an infusion bottle 143. Take three infusion pumps 142 for instance. As indicated in FIG. 6, when the first infusion pump 142 is turned on, the infusion device 140 performs a first stage of infusion with the first fluid; when the second the infusion pump 142 is turned on, the infusion device 140 performs a second stage of infusion with the second fluid; and when the third the infusion pump 142 is turned on, the infusion device 140 performs a third stage of infusion with the third fluid. Or, as indicated in FIG. 7, when the first infusion pump 142 is turned on, the infusion device 140 performs the first stage of infusion with the first fluid; when the second the infusion pump 142 and the third the infusion pump 142 are turned on, the infusion device 140 performs the second stage of infusion with the second fluid and the third fluid, and the rest can be obtained by analogy.

According to the infusion system and staged infusion method thereof disclosed in above embodiments of the present invention, the server can receive a patient information and a corresponding prescription information from a medical system and then transmit the prescription information to the infusion device located at the hospital bed according to the patient information. After verifying that the information is correct, the medical staff can turn on the infusion pump to perform staged infusion. Thus, the risk of having human errors during the manual input of infusion parameter can be avoided and labor cost can be saved.

While the invention has been described by way of example and in terms of the preferred embodiment(s), it is to be understood that the invention is not limited thereto. According to the technical features embodiments of the present invention, a person ordinarily skilled in the art will be able to make various modifications and similar arrangements and procedures without breaching the spirit and scope of protection of the invention. Therefore, the scope of protection of the present invention should be accorded with what is defined in the appended claims.

What is claimed is:

1. An infusion system, comprising:
a receiving module configured to receive a patient information and a corresponding prescription information from a medical system, wherein the prescription information comprises at least one fluid name, at least one fluid dose and a staged infusion parameter, and the patient information comprises a patient name and a corresponding position of a hospital bed;
a server connected to the receiving module to receive the prescription information and the patient information from the receiving module;
a transmission module connected to the server; and
an infusion device connected to the transmission module, wherein the transmission module serially connects the server and the infusion device and communicates with the server and the infusion device, so that the server transmits the prescription information to the infusion device located at the hospital bed according to the patient information,
wherein the infusion device displays the prescription information and the patient information and performs an infusion according to the staged infusion parameter.

2. The infusion system according to claim 1, wherein the staged infusion parameter comprises multiple staged infusion parameters, and the infusion device lists each of the staged infusion parameters according to the received prescription information.

3. The infusion system according to claim 2, wherein each staged infusion parameter comprises an infusion volume, an infusion rate and a time-interval between any two staged infusions; the infusion volume is equivalent to the product of the infusion rate multiplied by the infusion time.

4. The infusion system according to claim 3, wherein the infusion device comprises a management interface configured to control the infusion flow and the infusion rate, and a display interface configured to display the prescription information and the patient information.

5. The infusion system according to claim 1, wherein the infusion device may comprise an infusion pump, a plurality of infusion bottles and a plurality of infusion cannulas, the infusion bottles respectively contain a fluid, each of the infusion cannulas correspondingly connects one of the infusion bottles, and the infusion pump selectively connects one of the infusion cannulas to perform a staged infusion with one of the fluids.

6. The infusion system according to claim 1, wherein the infusion device comprises a plurality of infusion pumps, a plurality of infusion bottles and a plurality of infusion cannulas, the infusion bottles respectively contain a fluid, each of the infusion pumps correspondingly connects one of the infusion cannulas and one of the infusion bottles to perform a staged infusion with one of the fluids.

7. A staged infusion method configured in an infusion system, the staged
infusion method comprising:
receiving a patient information and a corresponding prescription information from a medical system by an infusion system, wherein the prescription information comprises at least one fluid name, at least one fluid dose and a staged infusion parameter, the patient information comprises a patient name and a corresponding position of a hospital bed;
transmitting the prescription information to an infusion device located at the hospital bed by the infusion system according to the patient information, wherein the infusion device displays the prescription information and the patient information; and
after verifying the prescription information and the patient information, informing the infusion device to perform a staged infusion according to the staged infusion parameter.

8. The staged infusion method according to claim 7, wherein performing
the staged infusion comprises:
starting a first stage of infusion, and performing a first staged infusion parameter, wherein the first staged infusion parameter comprises a first infusion volume and a first infusion rate;
determining whether the first stage of infusion is completed;
starting a second stage of infusion, and performing a second staged infusion parameter, wherein the second staged infusion parameter comprises a second infusion volume and a second infusion rate; and
determining whether the second stage of infusion is a last stage of infusion.

9. The staged infusion method according to claim 8, wherein the first infusion volume and the first infusion rate correspond to a first fluid, the second infusion volume and the second infusion rate correspond to a second fluid, the first fluid and the second fluid have different compositions, and a time-interval exists between the first stage of infusion and the second stage of infusion.

10. The staged infusion method according to claim 8, wherein the first infusion volume and the first infusion rate correspond to a first fluid, the second infusion volume and the second infusion rate correspond to a second fluid and a third fluid, wherein the first fluid, the second fluid and the third fluid have different compositions, and a time-interval exists between the first stage of infusion and the second stage of infusion.
